(19) United States
(12) Patent Application Publication
(10) Pub. No.: US 2020/0238067 A1
(43) Pub. Date: Jul. 30, 2020

(54) SELF-DEPLOYING INJECTOR FOR IMPLANTS AND METHODS OF MAKING AND USING SAME

(71) Applicant: DUKE UNIVERSITY, Durham, NC (US)

(72) Inventors: Mohamed IBRAHIM, Durham, NC (US); Mahmoud MOHAMMED, Durham, NC (US)

(21) Appl. No.: 16/635,935

(22) PCT Filed: Aug. 1, 2018
(86) PCT No.: PCT/US2018/044807

§ 371 Date: Jan. 31, 2020

Related U.S. Application Data
(60) Provisional application No. 62/539,595, filed on Aug. 1, 2017.

(51) Int. Cl. A61M 37/00; A61M 5/34; A61M 5/32

(52) U.S. Cl. CPC A61M 37/0069; A61M 2005/3151; A61M 5/3234; A61M 5/34

(57) ABSTRACT

Devices and methods for the injection of implants are provided, allowing for single-hand user implementation and activation. An implantation device comprises a body having a proximal end, a distal end, and a barrel extending therethrough. A needle extends through the distal end of the body and is in fluid communication with the barrel. An implant is positioned within the barrel for delivery to a subject, and a plunger, also positioned within the barrel, moves toward the distal end of the body so as to push against the implant and cause the implant to move in the distal direction, through the needle.

### CROSS-REFERENCE TO RELATED APPLICATIONS

This application claims priority to U.S. Provisional Patent Application Ser. No. 62/539,595 filed on Aug. 1, 2017, which is hereby incorporated by reference in its entirety.

### BACKGROUND

Implantable systems are used for a wide variety of applications. For example, implantable drug delivery systems provide for targeted, local delivery of a drug into a patient at a constant and predetermined rate. The implantation of a medicinal preparation subcutaneously is often used to achieve a long-term action for the preparation. The implant slowly releases a medical preparation, which is then transported into the patient's circulation via the blood and then further to a site within the patient's body to perform its intended action. Such implantable systems are thus useful for diseases requiring long-term therapy or when patient compliance issues arise. Because of the increasing importance of chronic diseases in human and veterinary medicine, long-lasting controlled release of active substance is of great interest. Other medical apparatuses, such as stents, filters, and sensors, may be delivered into a patient's body as implants as well.

However, the delivery of implants to a patient is often a difficult process. Maintaining control of a delivery device, ensuring the deployment of the implant into the correct anatomical plane, and placing the implant with minimal motional artifacts are all variables that result in implantation being a difficult procedure.

Currently, there is no established device that enables easy placement arid control for the precise placing of intradennal, subcutaneous and intramuscular implants.

### SUMMARY

In accordance with the present invention, systems and methods are defined for injecting an implant into a subject. In one example embodiment, an implantation device is provided. The device comprises a body having a proximal end, a distal end, and a barrel extending therethrough, a needle extending through the distal end of the body, and an implant positioned within the barrel for delivery to a subject. The device further comprises a plunger positioned within the barrel, a deployer that when activated applies force to the plunger to move the plunger toward the distal end of the body, and a friction member positioned within the barrel between the plunger and the distal end. The plunger is configured to contact the implant, pushing the implant through the needle and into a subject. When the plunger moves towards the distal end of the device, the plunger comes into contact with the friction member, thereby limiting the speed of the plunger.

In another example embodiment, a method is provided. The method serves to administer an active principle at a desired location in the body of a subject. The method comprises providing an implantation device, implanting the needle of the implantation device at the desired location in the body of the subject, and activating the plunger so as to move the plunger toward the active principle to force the active principle through the needle and into the body.

In another example embodiment, a kit for implanting a device into a patient is provided. The kit includes an implant delivery device and instructions for use of the implant delivery device. The implant delivery device comprises a body having a proximal end, a distal end, and a barrel extending therethrough, a needle extending through the distal end of the body, and an implant positioned within the barrel for delivery to a subject. The device further comprises a plunger positioned within the barrel, a deployer that when activated applies force to the plunger to move the plunger toward the distal end of the body, and a friction member positioned within the barrel between the plunger and the distal end. The implant delivery device may be preloaded in the kit.

These as well as other aspects and advantages of the synergy achieved by combining the various aspects of this technology, that while not previously disclosed, will become apparent to those of ordinary skill in the art by reading the following detailed description, with reference where appropriate to the accompanying drawings.

### BRIEF DESCRIPTION OF THE FIGURES

FIG. 1 depicts a cross-sectional view of an implantation device for deploying an implant into a patient, in accordance with at least one embodiment.

FIG. 2 depicts a needle for use with an implantation device, such as the implantation device of FIG. 1, in accordance with at least one embodiment.

FIG. 3 depicts an enlarged cross-sectional view of an example reservoir, such as the reservoir of FIG. 1, in accordance with at least one embodiment.

FIG. 4 depicts a cross-sectional view of an implantation device for deploying an implant into a patient, in accordance with at least one embodiment.

FIG. 4A depicts an enlarged view of a second activation mechanism of the implantation device of FIG. 4.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein. Unless otherwise defined, the technical terms used herein have the same meaning as commonly understood by one of ordinary skill in the art.

As used herein, the terms “subject” and “patient” are interchangeable and refer to both human and nonhuman animals. In certain embodiments, the subject is a human patient.

As used herein, the term “implant” refers to an injectable into a body of a subject. In some embodiments, an implant comprises an “active principle,” which as used herein comprises all substances capable of performing a medical fiinction, therapeutic or diagnostic. In some embodiments, an implant comprises a drug or a contraceptive capable of providing an immediate effect or a prolonged effect over time. In some embodiments, the implant comprises a single unit dose of a medication. In other embodiments, an implant comprises a hiosensor, a self-expanding polymeric stent, a filter, a polymeric embolic, or an occlusive implant. Still further medical injectables may be envisioned as comprising an implant for use with the implantation devices described herein.

### I. Overview

An implantation device is provided that enables a user to easily deploy an implant into a subject. The implantation device described herein may be used to deploy an implant intradermally, subcutaneously, or intramuscularly, for example. The implantation devices and systems, and associated methods of use described herein meet a clinical need for ease of placement and implantation of intradermal, subcutaneous, and intramuscular implants into a subject.

An implantation device comprises a body having a proximal end and a distal end and a barrel extending therethrough. A needle extends through the distal end of the body. The barrel of the body contains a plunger and a friction device therein, and near the proximal end of the body is a reservoir that contains a deploying member to apply a force to move the plunger toward the distal end of the body. Within the body and distal from the plunger is an implant for delivery through the needle into a subject.

The present disclosure advantageously provides a device allowing for single hand deployment of an implant into a subject. Improved control for implant delivery and ease of manipulation of a delivery device, instead of requiring use of a number of components to deliver an implant, is achieved. Further, the present disclosure advantageously minimizes accidental crushing of implanted material, which is a common occurrence using conventional methods for delivery of an implant into a patient.

### II. Example Embodiments

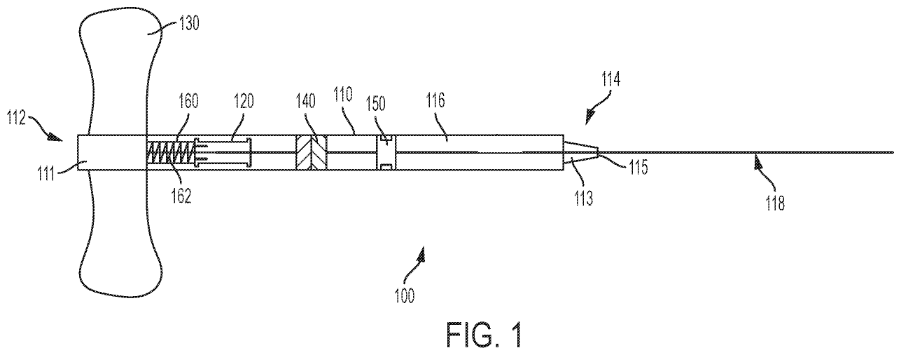

FIG. 1 depicts a cross-sectional view of an implantation device 100 for deploying an implant into a patient, in accordance with at least one embodiment.

The implantation device 100 comprises a body 110 having a proximal end 112, a distal end 114, and a barrel 116 extending therethrough. A needle 118 extends through the barrel opening at the distal end 114 and is in fluid communication with the barrel 116. An implant, such as any of the implants described herein, is pre-loaded in or near the tip of the needle 118.

The barrel 116 is formed of a tubular wall extending through the body interior. In some embodiments, the barrel 116 comprises a uniform diameter throughout the body 110. In other embodiments, the barrel 116 comprises a diameter that varies throughout the body. For example, the barrel diameter may comprise a smaller diameter in a section at or near the proximal end of the body, wherein a spring may be housed, than in a central portion of the body 110. In another example embodiment, the barrel diameter may decrease toward the distal end of the body, either in a tapered fashion or in a stepped fashion.

In some embodiments, the body 110 of the device 100 is generally cylindrical in shape, and may be manufactured by extruding a polymeric tube to define the body. The tube may be made from, for example, polyurethane or polyether block amide such as PEBAX®. In one example embodiment, an outer jacket 111 is applied over a section at or near the proximal end 112 of the body 110 as an extrusion of a material such as polyetheretherketone. In such an embodiment, the outer jacket 111 may extend across only a small portion of the body 110 near the proximal end or may extend across a larger portion of the body 110, including up to the distal end 114 of the body 110.

In some embodiments, the barrel 116 has a tapered or constricted portion 113 near or at distal end 114, and an outlet opening 115 at the distal end 114. At the proximal end 112, a grip 130 or knob may be provided for manipulation by a user. Near the proximal end 112 the barrel diameter is wide enough to receive a plunger 120.

The plunger 120 may be formed of any material suitable for use in a medical setting; for example, the plunger 120 may be formed of a hiocompatible plastic or polymer. As another example, the plunger 120 may be formed from stainless steel. As yet another example, the plunger 120 may comprise a material such as Nitinol. The plunger 120 comprises a shape sufficient to both fit within the inner diameter of the barrel portion and engage an implant. In some embodiments, the plunger 120 is mounted snugly for axial movement in the barrel 116. When force is applied to the plunger 120, the plunger 120 moves within the barrel in a direction toward the distal end 114. As discussed above, the plunger 120 is sized and shaped to engage the implant and to push the implant out of the distal end 114, through the needle 118, and ultimately into a subject.

Mounted within the barrel 116 is a friction member 150. The friction member 150 is positioned distal the plunger 120; that is, the friction member is located within the barrel 116 between the plunger 120 and the distal end 114 of the body 110. The friction member 150 is sized and shaped to fit within the barrel 116 of the body 110. The friction member serves to limit the speed of the plunger 120 as the plunger 120 moves toward the distal section 114.

The friction member 150 may comprise any of a number of materials that both grip the inner tubular wall 117 of the barrel 116 yet allow for movement of the member through the barrel 116. Example materials that may comprise the friction member include, but are not limited to, rubber and silicone. In certain embodiments, the friction member 150 comprises a rubber band.

In the embodiment shown in FIG. 1, the body 110 comprises a window 140 serving as an indicator of deployment of the device. In some embodiments, the window 140 comprises an opening into the barrel interior to allow a user to view the location of the plunger 120. In other embodiments, the window 140 comprises a type of visual indicator, such as a line or a pre-selected color, to show a user whether the device has been activated.

Mounted within the barrel 116 in the proximal section 112 of the device is a reservoir 160 comprising a deploying mechanism 162. When activated, the deploying mechanism 162 applies a force to the plunger 120. thereby moving the plunger 120 in the distal direction. In one example embodiment, the deploying mechanism 162 is a spring. In another example embodiment, the deploying mechanism 162 comprises a lever. Other forms of deployment mechanisms may be envisioned, and can include any mechanism able to release a force sufficient to move the plunger 120 down the length of the body 110. The deploying mechanism 162 may be manually activated by a user, or may be activated via an automated system, such as via a motor, or via a user-activated button.

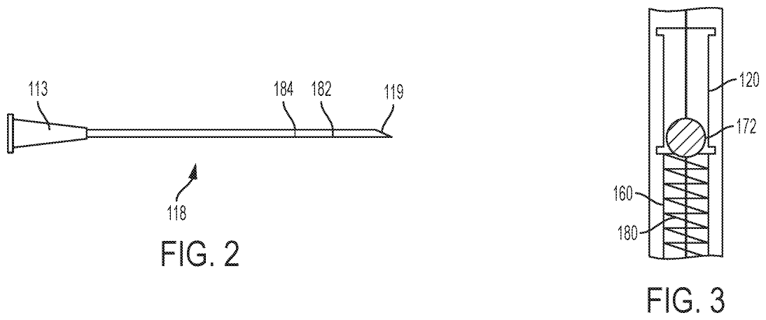

The needle 118 has both an inner diameter less than the inner diameter of the barrel 116 within the body 110 and an outer diameter that is less then the outer diameter of the body 110. The needle 118 comprises a sharp point or edge at its distal end 114, which is configured to pierce a subject's skin. The length and gauge of the needle 118 may be any of a number of lengths or gauges suitable for delivery of the implant. The length and gauge is dependent on numerous factors, including the size of the implant, the position of the delivery area in the subject, the toughness of material needed to insert the needle (e.g., skin or muscle). In some example embodiments, the gauge of the needle may range from about 7-34. In certain embodiments, the needle gauge ranges from about 21-27. Similarly, the length of the needle may comprise any length suitable for the delivery of the implant. In some example embodiments, the length of the needle may range from about 1-20 cm. In certain embodiments, the needle length comprises a length of about 2-12 cm. FIG. 2 depicts a needle for use with an implant device, such as the device 100 of FIG. 1, in accordance with at least one embodiment. The tapered portion 115 of the body 110 of the device 100 is shown connected to the needle 118. The needle 118 is shown comprising a beveled tip 119, which aids in the insertion of the needle 118 into a subject. Also shown in FIG. 2 is a first marking 182 which serves as an insertion guiding mark, and a second marking 184 which serves as an insertion stop mark to ensure that the device is not inserted past a given depth into a subject.

In some embodiments, there may be more than one first marking 184 indicating depth of the needle into a patient. For example, a plurality of markings may indicate depth in millimeters, centimeters, inches, or the like of the needle into a patient.

In some embodiments, the second marking 184 comprises a physical barrier affixed to the needle that physically prevents the needle from further insertion into a patient. In such an embodiment, the physical barrier may be user adjustable (e.g., able to slide along the needle length) to allow a user to determine, prior to insertion, where the stop is desired for a given insertion.

FIG. 3 depicts an enlarged view of an example reservoir, such as the reservoir 160 in FIG. 1, in accordance with at least one embodiment. In FIG. 3, a button 172 serves to activate the deploying mechanism when depressed. In operation, the spring 180 is held in a compressed state, in tension, by the button 172. The button 172 physically blocks or is connected to a mechanism which physically maintains the spring 180 in its compressed state or otherwise prevents the spring 180 from achieving its extended length. When a user presses the button 172 in a direction toward the device, the button 172 movement removes the blockage, releasing tension on the spring and allowing the spring to extend. The spring 180 then transitions from a compressed state to an extended state in the distal direction, in turn pushing the plunger 120 in a direction toward the distal end 114 of the device.

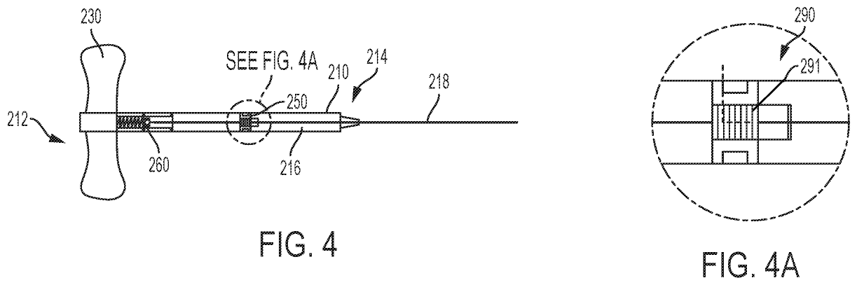

In some embodiments, an implantation device comprises a second activation mechanism. FIG. 4 depicts a cross-sectional view of an implantation device 200 for deploying an implant into a patient, in accordance with at least one embodiment. Similar to the device 100 of FIG. 1, the implantation device 200 has a proximal end 212, a distal end 214, and a barrel 216 extending therethrough. A needle 218 extends through the distal end 214 and is in fluid communication with the barrel 216.

A second activation mechanism 290 is shown positioned in the body 210 of the device and comprises a second deploying mechanism positioned at or near the friction member 250. The second activation mechanism 290 may include a button that operates in the same manner or similar to the first activation mechanism 260. FIG. 4A depicts an enlarged view of a second activation mechanism of the implantation device of FIG. 4.

When activated, the second activation mechanism 290 releases a second deploying mechanism 291. in one example embodiment, the second deploying mechanism 291 comprises a spring. In this embodiment, one of either the first or second activation mechanisms may be configured for automatic deployment, and the other of the first or second activation mechanisms may be configured for manual deployment. in one example embodiment, automatic deployment comprises a button that, when depressed, automatically deploys a spring to apply force to an implant. In one example embodiment, manual deployment comprises a manual button that requires more of a sliding three to activate force that will then be applied to an implant. In some embodiments, both the first and second activation mechanisms are configured for automatic deployment or for manual deployment.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims, along with the full scope of equivalents to which such claims are entitled. It is also to be understood that the terminology used herein is for the purpose of describing particular embodiments only, and is not intended to be limiting.

1. An implantation device comprising:
a body having a proximal end, a distal end, and a barrel extending therethrough;
a needle extending through the distal end of the body;
an implant positioned within the barrel for delivery to a subject;
a plunger positioned within the barrel;
a first deployer that when activated applies force to the plunger to move the plunger toward the distal end of the body; and
a friction member positioned within the barrel between the plunger and the distal end,
wherein the plunger is configured to contact the friction member, thereby limiting the speed of the plunger when the plunger moves towards the distal end, and wherein the plunger is configured to contact the implant.

2. The implantation device of claim 1, wherein when the plunger moves toward the distal end, the plunger is configured to apply sufficient force on the implant to move the implant distally through the needle and into a subject.

3. The implantation device of claim 1, wherein the first deployer is a spring having an initial compressed state.

4. The implantation device of claim 3, the body further comprising a first releasable retaining mechanism to bias the spring in the initial compressed state.

5. The implantation device of claim 1, wherein the first releasable retaining mechanism is a button that, when depressed, releases a barrier holding the spring in the initial compressed state, thereby allowing for movement of the spring toward the distal end of the body.

6. The implantation device of claim 5, wherein as the spring moves from the initial compressed state to an extended state, the spring contacts the plunger, applying the force to the plunger to move the plunger toward the distal end of the body.

7. The implantation device of claim 1, further comprising a second deployer and a second retaining mechanism, both positioned distal from the first deployer and the first retaining mechanism.

8. The implantation device of claim 7, wherein the second deployer is a second spring and the second retaining mechanism comprises a button, that when depressed removes a barrier preventing the second spring from movement.

9. The implantation device of claim 1, wherein the needle is connected to the body via a needle carrier affixed to the distal end of the body, and wherein the needle is a beveled needle.

10. The implantation device of claim 1, wherein the needle comprises markings to guide insertion and deployment distance.

11. The implantation device of claim 10, wherein the markings comprise one or more insertion guidance marks that serve to indicate location for insertion into the subject or provide information (e.g., numerical markings) to a user regarding the needle depth into a patient, and a stop mark indicating a maximum depth for insertion of the needle into the subject.

12. The implantation device of claim 1, wherein the friction mechanism is a rubber band positioned adjacent the inner wall of the barrel.

13. The implantation device of claim 1, further comprising a status window on the body indicating deployment of the device.

14. A method of administering an active principle at a desired location in the body of a subject comprising:
providing the implantation device of claim 1;
implanting the needle of the implantation device at the desired location in the body of the subject; and
activating the plunger so as to move the plunger toward the active principle to deliver the active principle through the needle and into the body.

15. The method of claim 14, wherein activating the plunger further comprises:
depressing a first button located on an exterior surface of the body.

16. The method of claim 15, wherein activating the plunger further comprises:
depressing a second button located on an exterior surface of the body.

17. The method of claim 14, wherein implanting the needle of the implantation device further comprises:
implanting the needle up to a marking indicating a pre-determined depth for insertion into a subject.

18. A kit for implanting a device into a patient comprising:
the implant delivery device of claim 1; and
instructions for use of the implant delivery device.

19. The kit of claim 18, wherein the instructions for use comprise the method of administering an active principle at a desired location in the body of a subject comprising:
providing the implantation device;
implanting the needle of the implantation device at the desired location in the body of the subject and
activating the plunger so as to move the plunger toward the active principle to deliver the active principle through the needle and into the body.
